(19) United States
(12) Patent Application Publication
(10) Pub. No.: US 2026/0355274 A1
(43) Pub. Date: Nov. 12, 2026

(54) HDAC INHIBITOR OKI-179 FOR THE TREATMENT OF CANCERS RESULTING FROM A MAPK PATHWAY MUTATION

(71) Applicant: Onkure, Inc., Boulder, CO (US)

(72) Inventors: Duncan Walker, Boulder, CO (US); Richard D. Woessner, Boulder, CO (US)

(73) Assignee: Onkure, Inc., Boulder, CO (US)

(21) Appl. No.: 19/110,664

(22) PCT Filed: Sep. 14, 2023
(86) PCT No.: PCT/US2023/032682

§ 371 Date: Mar. 11, 2025

Related U.S. Application Data
(60) Provisional application No. 63/408,323, filed on Sep. 20, 2022.

(51) Int. Cl. A61K 38/15; A61K 45/06; A61P 35/00

(52) U.S. Cl. CPC A61K 38/15; A61K 45/06; A61P 35/00

(57) ABSTRACT

OKI-179 alone or in combination with a MAPK pathway inhibitor is described for the treatment of a cancer resulting from one or more mutations in the MAPK pathway.

### CROSS REFERENCE TO RELATED APPLICATIONS

This application claims priority to U.S. Provisional Application No. 63/408,323, filed Sep. 20, 2022, the disclosure of which is incorporated in its entirety for all purposes.

### BACKGROUND OF THE INVENTION

It is well known that the RAS pathway is frequently activated in human cancers and thus may provide multiple opportunities for therapeutic interventions. Activated growth factor receptors signal through RAS, as well as other pathways, and RAS mutations account for 50% or more of colorectal cancers, lung cancers and significant fractions of other cancers (Sanchez-Vega et al., Cell. 173(2):321-337.e10. doi: 10.1016/j.cell.2018.03.035. PMID: 29625050; PMCID: PMC6070353 (2018). Activating RAS pathway mutations are found in greater than 30% of human tumors and are often associated with poor outcomes. While RAS-pathway targeted drugs have been approved, their activities as single agents remain modest, thus emphasizing the need for rational targeted combinations to improve patient outcomes.

Multiple studies have proposed a chemical synthetic lethality or synergistic interaction between Class I histone deacetylase inhibitors (HDACI), and RAS-pathway inhibitors in RAS-pathway mutated models (Maertens et al., Cancer Discovery 9, 526-545 (2019); Yamada et al., Mol. Cancer Ther. 17, 17-25 (2018); Faiao-Flores et al., Melanoma Manag. 6 (4): MMT29. doi: 10.2217/mmt-2019-0017 (2019); Ischenko et al., Oncotarget. 6 (18), 15814-27 (2015); Wang et al., Cell 173, 1413-25 (2018); Chao et al., Clin Epigenetics 11 (1): 85. doi: 10.1186/s13148-019-0681-6 (2019); Bahr et al., Oncotarget 7 (43), 69804-69815 (2016)). This chemical synthetic lethality occurs due to a drug combination effect and is proposed to result in the inhibition of double-stranded (ds) DNA repair and other survival pathways which drive apoptosis and tumor regressions (Maertens et al., Cancer Discovery 9, 526-545 (2019)). The use of HDACi in solid tumors and in combination therapies has been impeded by poor potency, lack of selectivity, safety issues and dosing inconvenience, thus highlighting the need for a better HDACi in the clinic.

OKI-179 is a novel largazole derivative and a potent Class I-selective, oral HDAC inhibitor that has completed Phase 1 clinical trials as a single agent in patients with solid tumors. The clinical profile of OKI-179 shows its potential to achieve exposure consistent with preclinical activity, with strong pharmacodynamic activity at tolerated doses, supporting the development of OKI-179 for use in solid tumor combinations. Notably, OKI-179 addresses historic limitations associated with prior HDAC inhibitors by showing improved selectivity, improved tolerability and bioavailability, and a clear development path in a molecularly targeted population.

### SUMMARY OF THE INVENTION

An aspect of the invention is a pharmaceutical composition for the treatment of cancer, the composition comprising OKI-179 and a pharmaceutically acceptable carrier, wherein the cancer harbors one or more mutations that results in activation of the mitogen-activated protein kinase (MAPK) pathway.

Another aspect of the invention is a pharmaceutical combination for the treatment of cancer, the combination comprising:
OKI-179; and
one or more mitogen-activated protein kinase (MAPK) pathway inhibitors,
wherein the cancer harbors one or more mutations that results in activation of the MAPK pathway.

Another aspect of the invention is a pharmaceutical combination, the combination comprising:
OKI-179; and
one or more mitogen-activated protein kinase (MAPK) pathway inhibitors.

Another aspect of the invention is a method of treating cancer in a subject comprising administering to the subject in need thereof a therapeutically effective amount of OKI-179, wherein the cancer harbors one or more mutations that results in activation of the MAPK pathway.

Another aspect of the invention is a method of treating cancer in a subject comprising administering to the subject in need thereof therapeutically effective amounts of
OKI-179; and
one or more mitogen-activated protein kinase (MAPK) pathway inhibitors,
wherein the cancer harbors one or more mutations that results in activation of the MAPK pathway.

In an exemplary embodiment, the one or more MAPK pathway inhibitors is selected from an EGFR inhibitor, a RAS inhibitor, a BRAF inhibitor, a pan-RAF inhibitor, a MEK inhibitor and an ERK inhibitor.

In an exemplary embodiment, administration of the OKI-179 and the one or more MAPK pathway inhibitors in combination to a subject in need thereof results in synergistic inhibition of cancer growth.

In an exemplary embodiment, the mutation occurs in one or more of EGFR, NF1, RAS, BRAF, MAP2K1, GNAQ and GNA11.

In an exemplary embodiment, the one or more MAPK pathway inhibitors comprises or consists of an EGFR inhibitor.

In an exemplary embodiment, the one or more MAPK pathway inhibitors comprises or consists of a RAS inhibitor.

In an exemplary embodiment, the one or more MAPK pathway inhibitors comprises or consists of a BRAF inhibitor.

In an exemplary embodiment, the one or more MAPK pathway inhibitors comprises or consists of a pan-RAF inhibitor.

In an exemplary embodiment, the one or more MAPK pathway inhibitors comprises or consists of a MEK inhibitor.

In an exemplary embodiment, the one or more MAPK pathway inhibitors comprises or consists of an ERK inhibitor.

In an exemplary embodiment, the mutation occurs in two or more of EGFR, NF1, RAS, BRAF, MAP2K1, GNAQ and GNA11.

In an exemplary embodiment, the mutation occurs in three or more of EGFR, NF1, RAS, BRAF, MAP2K1, GNAQ and GNA11.

In an exemplary embodiment, the mutation occurs in RAS (e.g., KRAS, HRAS or NRAS) alone or in combination with other mutations that include one or more of EGFR, NF1, BRAF, MAP2K1, GNAQ and GNA11.

In an exemplary embodiment, the mutation occurs in EGFR alone or in combination with other mutations that include one or more of NF1, RAS, BRAF, MAP2K1, GNAQ and GNA11.

In an exemplary embodiment, the mutation occurs in NF1 alone or in combination with other mutations that include one or more of EGFR, RAS, BRAF, MAP2K1, GNAQ and GNA11.

In an exemplary embodiment, the mutation occurs in BRAF alone or in combination with other mutations that include one or more of EGFR, NF1, RAS, MAP2K1, GNAQ and GNA11.

In an exemplary embodiment, the mutation occurs in MAP2K1 alone or in combination with other mutations that include one or more of EGFR, NF1, RAS, BRAF, GNAQ and GNA11.

In an exemplary embodiment, the mutation occurs in GNAQ alone or in combination with other mutations that include one or more of EGFR, NF1, RAS, BRAF, MAP2K1 and GNA11.

In an exemplary embodiment, the mutation occurs in GNA11 alone or in combination with other mutations that include one or more of EGFR, NF1, RAS, BRAF, MAP2K1 and GNAQ.

In an exemplary embodiment, the combination is not OKI-179 and binimetinib.

In an exemplary embodiment, the combination is not OKI-179, binimetinib and encorafenib.

In an exemplary embodiment, the cancer is colorectal cancer.

In an exemplary embodiment, the cancer is breast cancer.

In an exemplary embodiment, the cancer is non-small cell lung cancer (NSCLC).

In an exemplary embodiment, the cancer is melanoma.

In an exemplary embodiment, the at least one MAPK pathway inhibitor and the OKI-179 of

the composition are administered concurrently.

In an exemplary embodiment, the at least one MAPK pathway inhibitor and the OKI-179 of the composition are administered sequentially.

### BRIEF DESCRIPTION OF THE DRAWINGS

The following figures are illustrative of specific embodiments of the invention and are not intended to otherwise limit the scope of the invention as described herein. OKI-005 is an in vitro optimized tool compound. Both OKI-005 and OKI-179 are metabolized to the same active compound, OKI-006. In the figures, “bini” is equivalent to binimetinib (a MEK 1/2 inhibitor) and “enco” is equivalent to encorafenib (a BRAF inhibitor).

FIGS. 1A and 1B illustrate how targeting RAS-pathway mutations (such as BRAF, KRAS^{G12C} and NF1) (FIG. 1A) have changed the approach in treating cancers such as the listed melanoma, colon, lung, neurofibromatosis, thyroid, tumor agnostic and pediatric cancers and the identified drug(s) used to treat these cancers with the indicated overall response rates (ORR) and progression-free survival (PFS) values (shown as median overall survival (mOS) versus control) (FIG. 18).

FIG. 2 illustrates that despite the success shown in FIG. 1B, RAS pathway targeting is not particularly effective in some indications, and even in “sensitive” indications, a significant proportion of patients (illustrated by the shaded regions) do not receive a clinical benefit from single-agent RAS pathway inhibition. The relative ineffectiveness of the BRAF^{V600E} kinase inhibitor vemurafenib against BRAF^{V600E} melanoma and BRAF^{V600E} colorectal cancer (CRC); and the relative ineffectiveness of the KRAS^{G12C} kinase inhibitor sotorasib against KRAS^{G12C} pancreatic cancer and KRAS^{G12C} colorectal cancer (CRC) are shown as specific examples.

FIG. 3 illustrates chemical synthetic lethality as an approach to improving RAS-pathway targeting by driving rapid cell death by focusing on pathways necessary for cell survival following inhibition of mutated RAS-pathway signaling. This approach is based on the biological dependence on RAS-pathway mutations and on rapid onset of cell death which avoids compensatory RAS-pathway reactivation and resistance. Exemplary employed chemical agents include histone deacetylase (HDAC) inhibitors.

FIG. 4 illustrates the potential for synthetic lethal interaction between RAS/mitogen-activated protein kinase (MAPK) inhibition and targeting non-homologous end joining (NHEJ) and homologous recombination repair (HRR) in RAS-mutated tumors as described in Kalimutho et al., Molec. Oncol. 5, 470-90 (2017). The RAS pathway can upregulate HRR as a mechanism to compensate for increased ds-DNA damage. The RAS/MAPK pathway inhibition in RAS-pathway mutated cells can downregulate HRR, creating a dependence on NHEJ. Thus, there is a potential for synthetic lethal interaction between RAS/MAPK inhibition and targeting NHEJ/HRR in RAS-mutated tumors. In the Figure, DDR is DNA-damage response, PARP is poly-ADP ribose polymerase, and MYC is myelocytomatosis oncogene.

FIG. 5 illustrates that tolerated AUC (area under the curve) levels cover efficacious exposure in animal (mouse) xenograft models at the indicated dosage levels of OKI-179 and shows that synergy associated with OKI-179 and binimetinib occurs at physiologically-relevant doses of OKI-179. MTD is maximal tolerated dose. The Nautilus Ph2 dose has been clinically determined to reflect safe and efficacious ranges of OKI-179 combined with binimetinib in patients with NRAS-mutated melanoma, where OKI-179 is administered on a 4 days on/3 days off dosing schedule.

FIGS. 6A, 6B and 6C illustrate that OKI-005 combined with binimetinib shows synergistic anti-proliferative activity in cell lines harboring RAS mutations. Synergy was observed in models harboring NRAS (FIG. 6A—SKMEL2 melanoma cell line) and KRAS (FIG. 68—AGS human gastric adenocarcinoma hyperdiploid cell line and FIG. 6C—NCI-H358 non-small cell lung cancer cell line) mutations. In particular, cells are grown in multi-well plates and incubated with the OKI-005 and binimetinib for 72 hours. The number of viable cells is determined by luminescence readout after the addition of CellTiter-Glo (CTG) dye.

FIGS. 7A and 7B illustrate that OKI-005 combined with binimetinib shows synergistic anti-proliferative activity in combination in human uveal melanoma cell lines (92-1 and MP46) harboring a GNAQ/11 Q209L mutation. In particular, cells are grown in multi-well plates and incubated with the OKI-005 and binimetinib for 72 hours. The number of viable cells is determined by luminescence readout after the addition of CellTiter-Glo (CTG) dye.

FIG. 8 illustrates that in a 72-hour proliferation assay at physiologically relevant concentrations of OKI-005 and binimetinib (where a value of <1 indicates a net cell loss), there was observed synergy associated with this combination in the RAS mutated pathway (i.e., a superior effect greater than that of either agent alone) compared to the RAS wild-type (WT) pathway after Day 3 compared to Day 0 as determined by luminescence readout after the addition of CellTiter-Glo (CTG) dye. The RAS mutated cell lines and the RAS WT cell lines involved in this study represent conventional and well known cell lines for this purpose. See, e.g., FIG. 16.

FIGS. 9A, 9B and 9C illustrate that the synergy associated with the combination of OKI-005 and binimetinib (in the indicated amounts) in the NRAS-mutated SKMEL2 cell line is associated with significant cell growth inhibition and cell death as measured by PARP cleavage. In particular, in FIGS. 9A and 9B, cells are grown in multi-well plates and incubated with the OKI-005 and binimetinib for 72 hours. The number of viable cells is determined by luminescence readout after the addition of CellTiter-Glo (CTG) dye. SEM is the standard error of the mean,

FIG. 10 illustrates the synergistic effects in O⁶-methylguanine-DNA methyltransferase (MGMT)-positive human tumor cell lines versus MGMT-negative human tumor cell lines (“MGMT Null”) when dosed with the combination of OKI-005 and binimetinib compared to OKI-005 or binimetinib alone.

FIGS. 11A, 11B and 11C illustrate the administration of OKI-179 and binimetinib at doses and schedules that model clinically approved or tolerated exposures in the NRAS mutated cell line xenograft SKMEL2. As used herein, “Son/2off” refers to a dosing schedule of 5 days on and 2 days off each week. Changes in the SKMEL2 tumors pERK (MEKi PD) and Ac-H3K9 (HDACI PD) were observed at 2 hours post dose. Glyceraldehyde 3-phosphate dehydrogenase (GAPDH) was used as a control. Both binimetinib and OKI-179 showed strong on-target PD activity (i.e., increased AC-H3K9 or decreased pERK) at the indicated doses. OKI-179 or binimetinib as single agents showed tumor growth delay, but few regressions. OKI-179 in combination with binimetinib showed significantly increased regressions compared to either single agent following two weeks of dosing.

FIGS. 12A, 12B and 12C illustrate the results of studies of the combination of OKI-005 with each of binimetinib, GDC-0994 and sotorasib in the NCI-H358 (NSCLC KRAS^{G12C}) cell line. FIGS. 12D, 12E and 12F illustrate the results of studies of the combination of OKI-005 with each of binimetinib, GDC-0994 and MRTX1133 (a KRAS^{G12D} Inhibitor) in the AGS (Gastric KRAS^{G12D}) cell line. By Loewe synergy analysis, combination synergy was observed in all cases. In particular, cells are grown in multi-well plates and incubated with the OKI-005 and binimetinib for 72 hours. The number of viable cells is determined by luminescence readout after the addition of CellTiter-Glo (CTG) dye.

FIG. 13A illustrates that OKI-179 combined with binimetinib and encorafenib in HT29 BRAF^{V600E} colorectal (CRC) xenografts using the indicated amounts and dosing regimens of each agent (mpk=mg/kg) shows significantly increased regressions compared to either OKI-179 alone or the combination of binimetinib and encorafenib following two weeks of dosing. FIG. 13B illustrates the comparative mouse body weight changes for OKI-179 alone, the combination of binimetinib and encorafenib, and OKI-179 combined with binimetinib and encorafenib at the indicated doses and up to 15 days after randomization. It was observed that OKI-179 combined with binimetinib and encorafenib resulted in greater weight loss than either OKI-179 alone or the combination of binimetinib and encorafenib.

FIG. 14A illustrates that OKI-179 combined with sotorasib in NCI-H358 KRAS^{G12C} non-small cell lung cancer xenografts shows significantly increased regressions compared to either OKI-179 or sotorasib alone over a 50-day period after dosing at the indicated levels and rates. As used herein, “4on/3off” refers to a dosing schedule of 4 days on and 3 days off each week. FIG. 14B illustrates mouse body weight changes for OKI-179 and sotorasib alone compared to the combination of OKI-179 with sotorasib.

FIG. 15 illustrates by tumor type of the listed conventional cell lines the effectiveness of the combination of OKI-005 and binimetinib compared to OKI-005 and binimetinib alone at the indicated dose levels as measured by a fold change in the CTG signal 3 days after dosing.

FIG. 16 illustrates by MAPK mutation of the listed conventional tumor cell lines the effectiveness of the combination of OKI-005 and binimetinib compared to OKI-005 and binimetinib alone at the indicated dose levels as measured by a fold change in the CTG signal 3 days after dosing.

FIG. 17 illustrates the effectiveness against the listed conventional tumor cell lines of the combination of OKI-005 and binimetinib compared to the combination of OKI-005 and the ERK 1/2 inhibitor GDC-0994 sorted by the binimetinib response as measured by a fold change in the CTG signal 3 days after dosing.

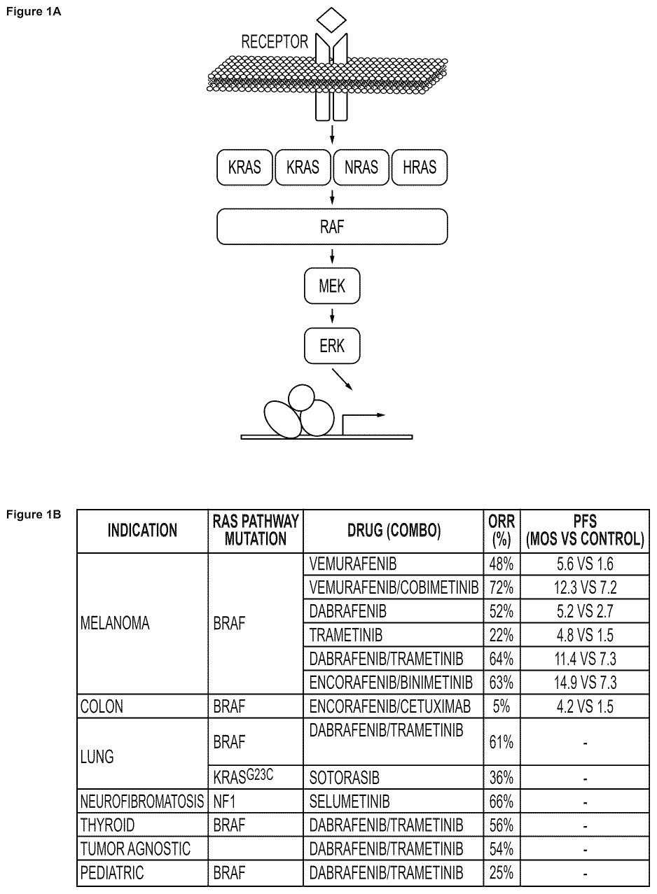

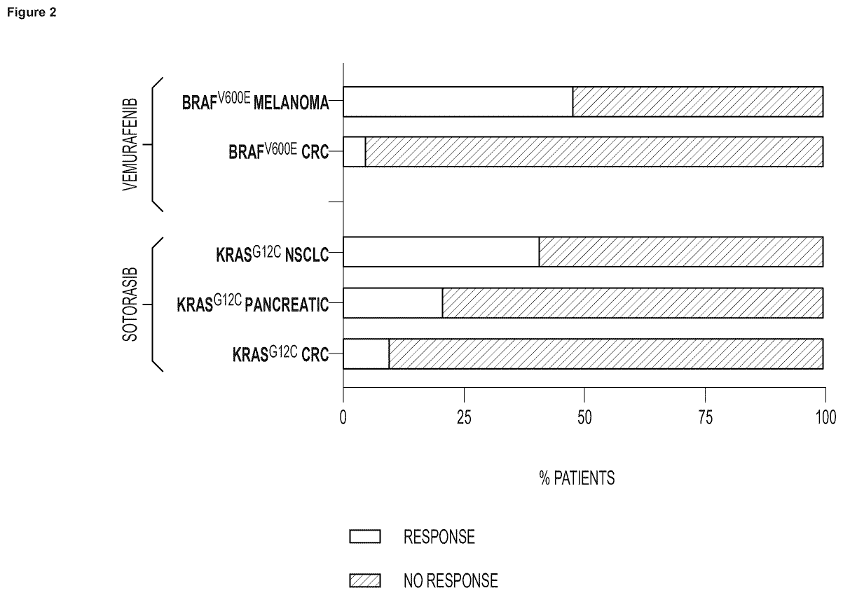

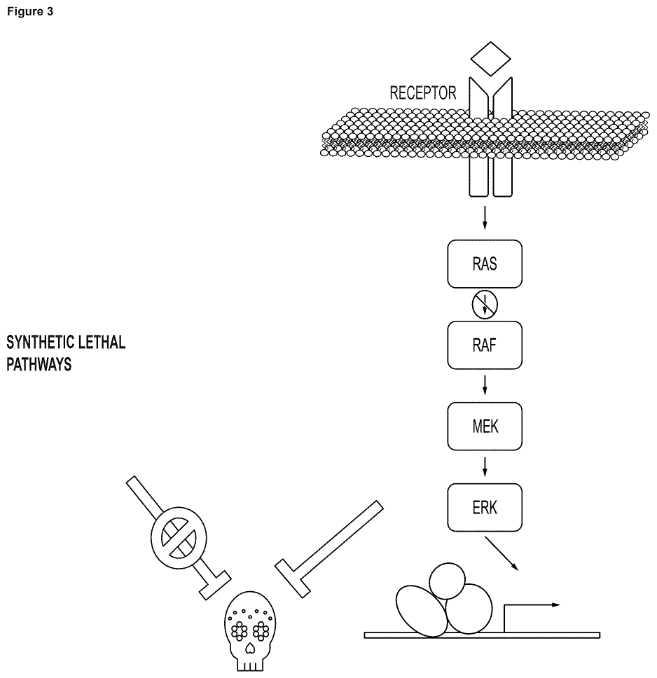

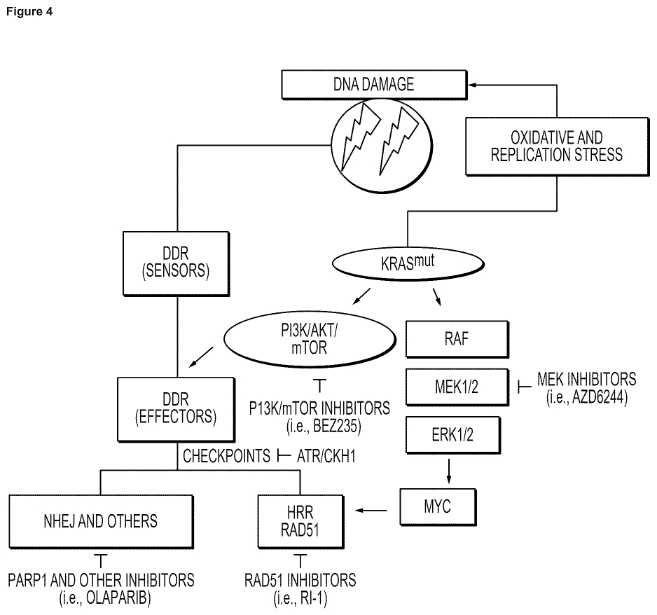

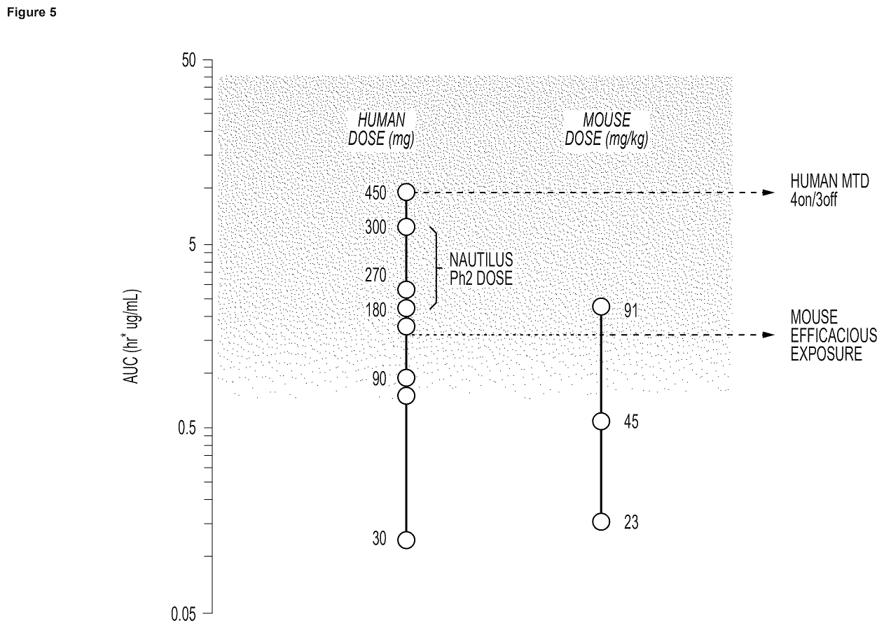

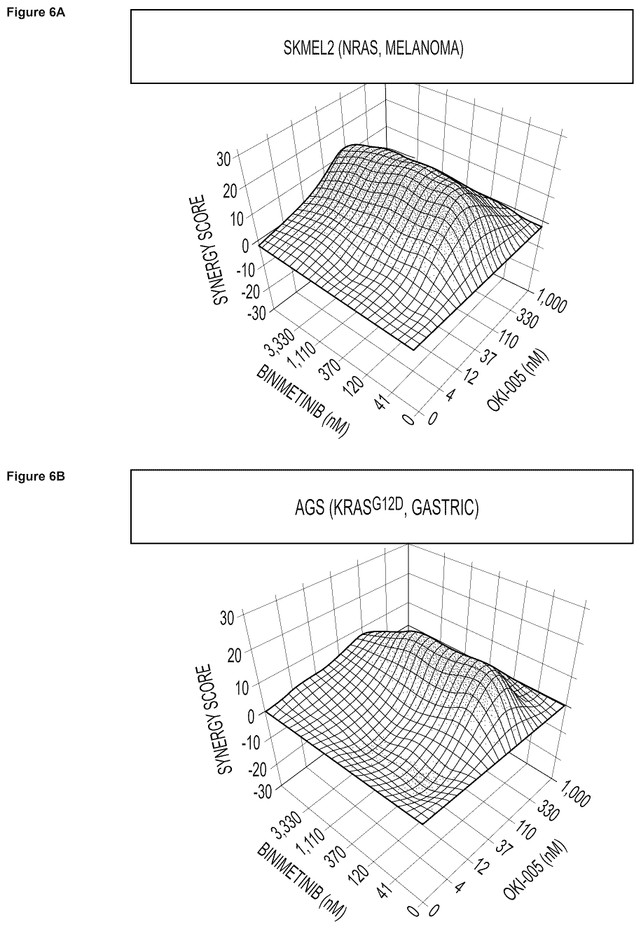

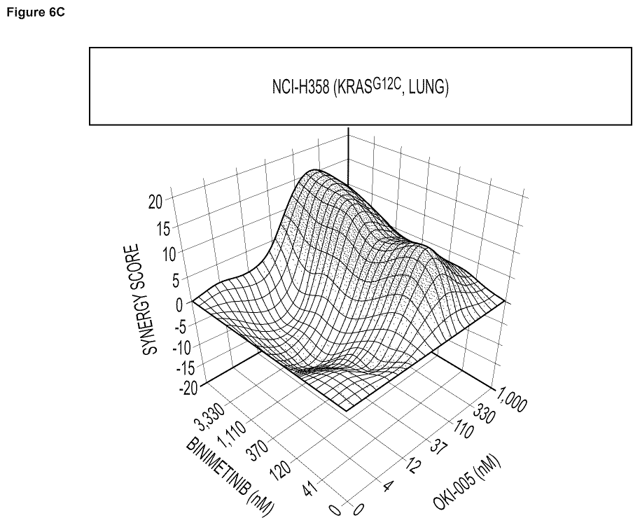

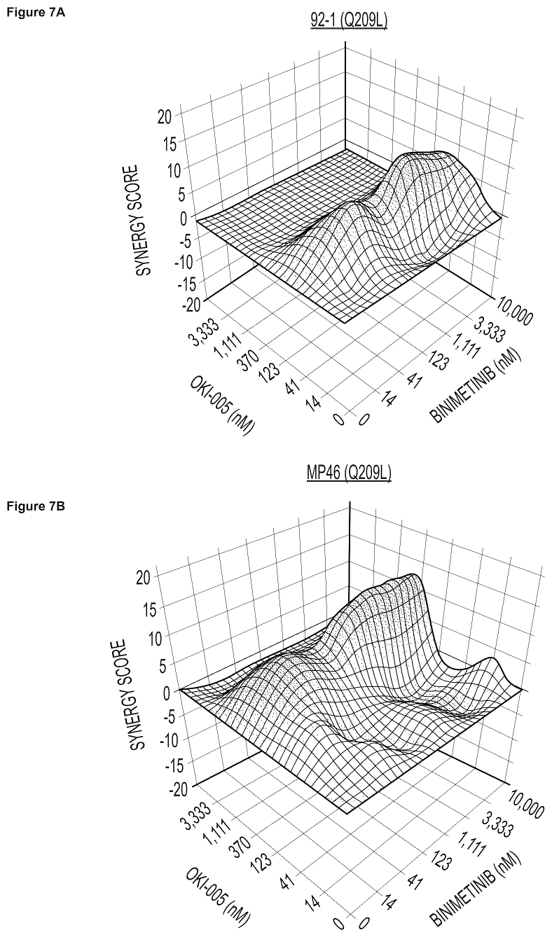

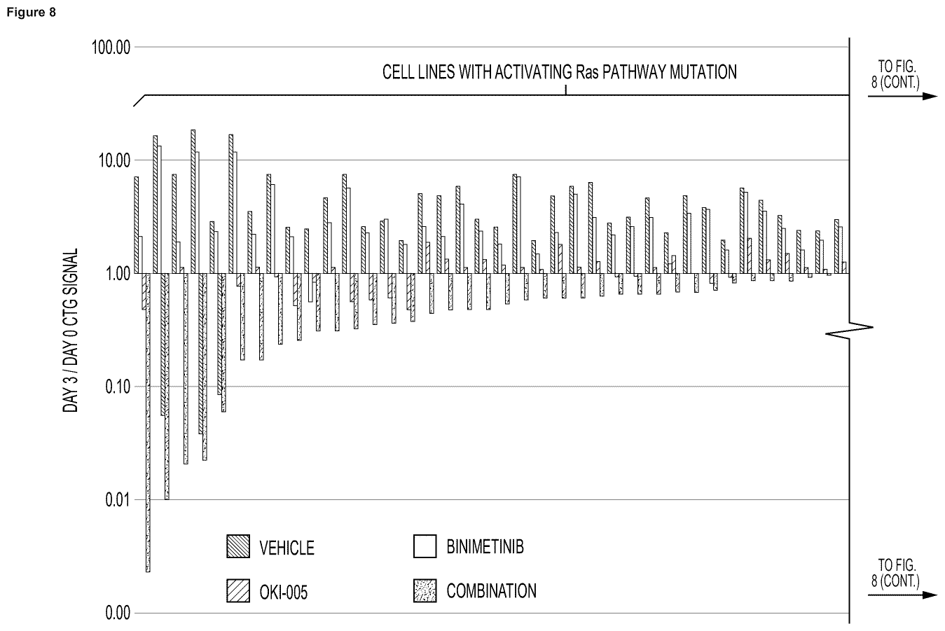

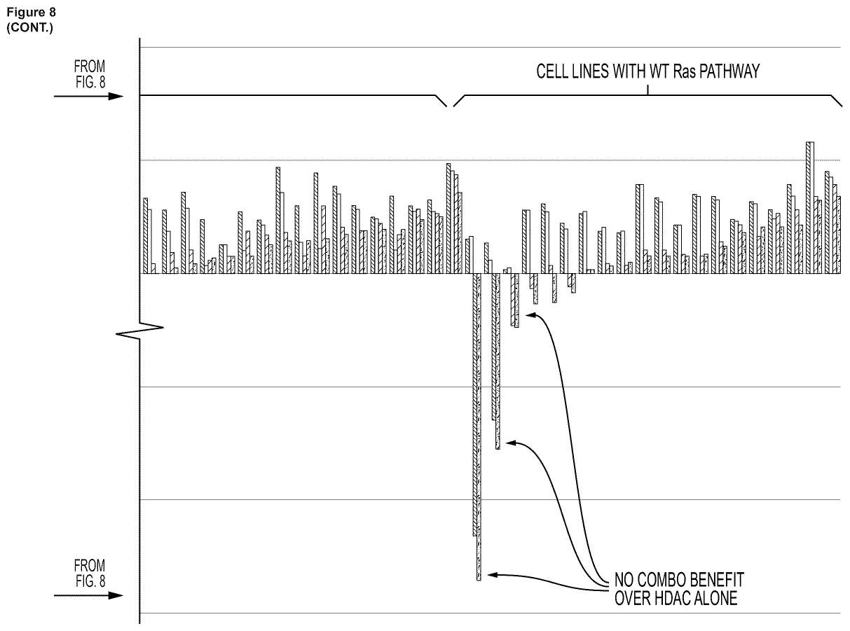

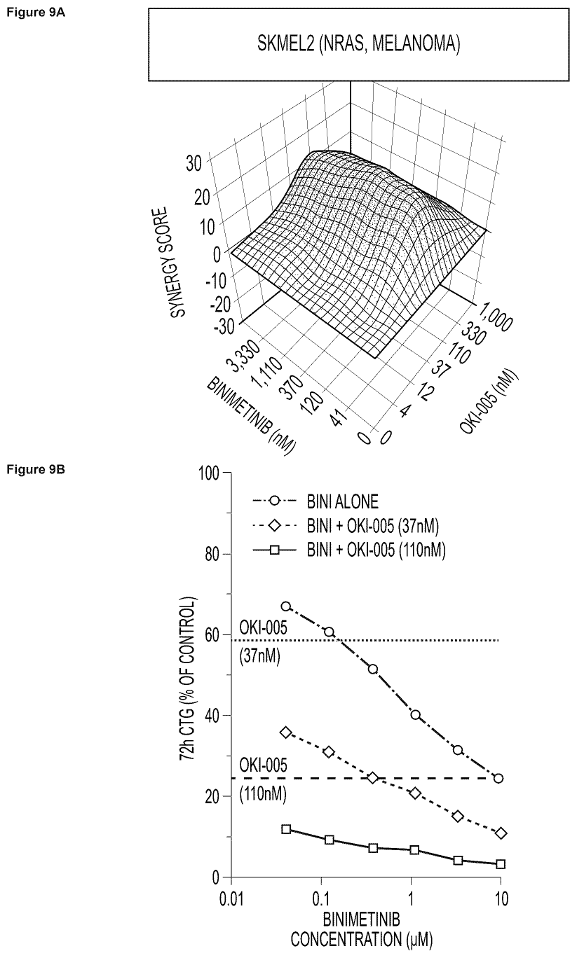

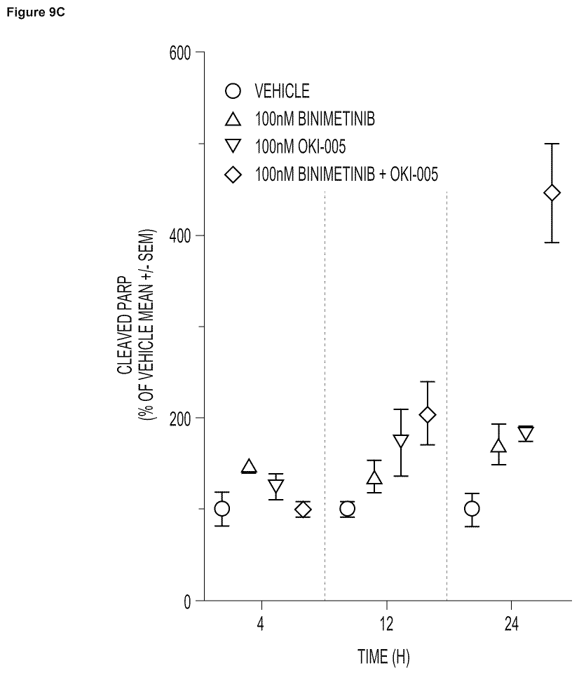

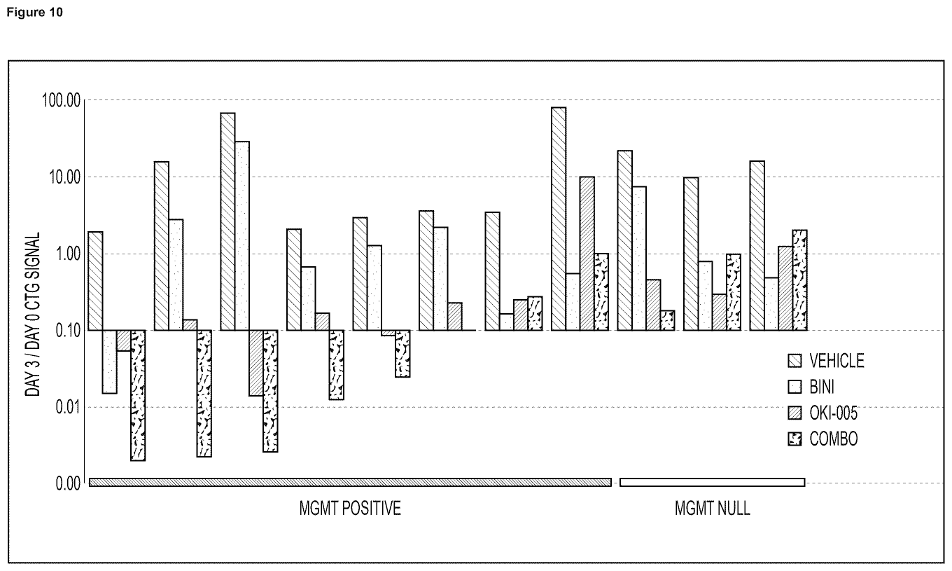

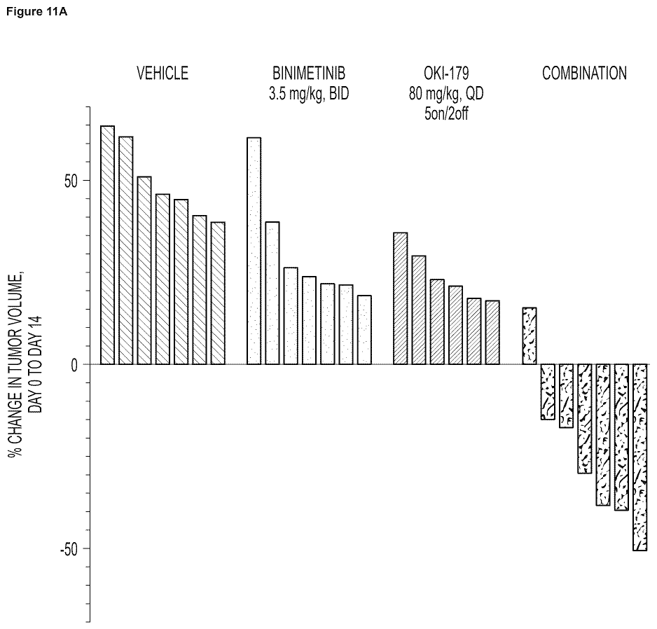

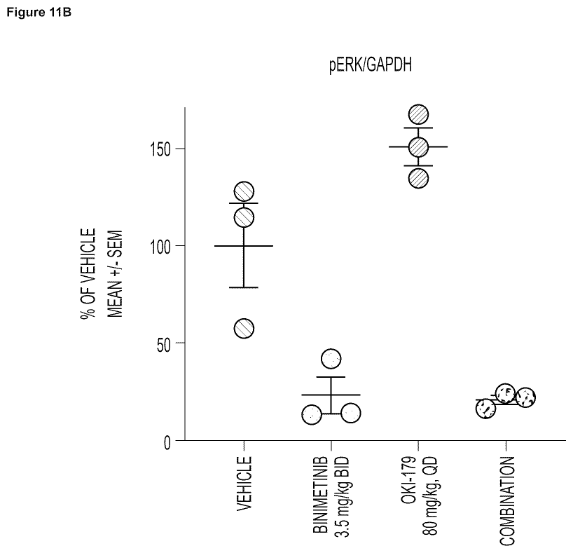

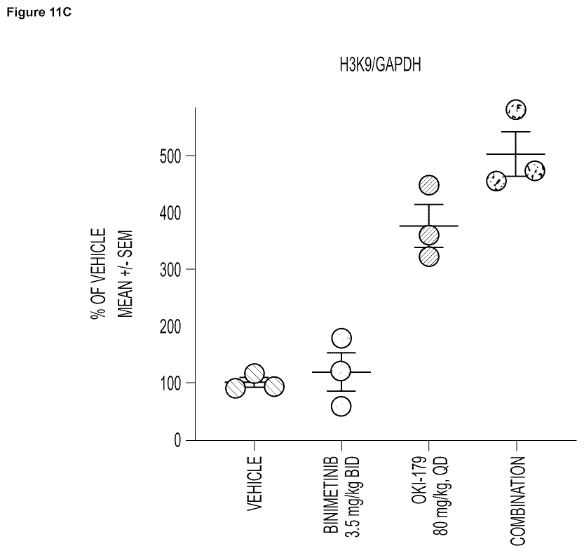

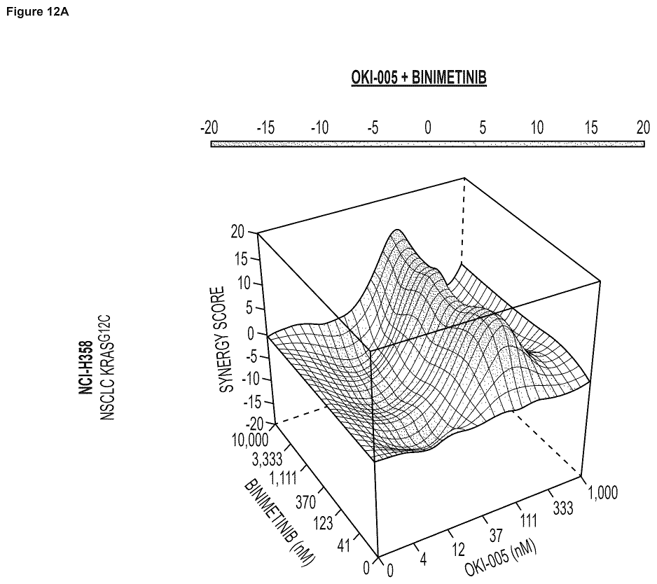

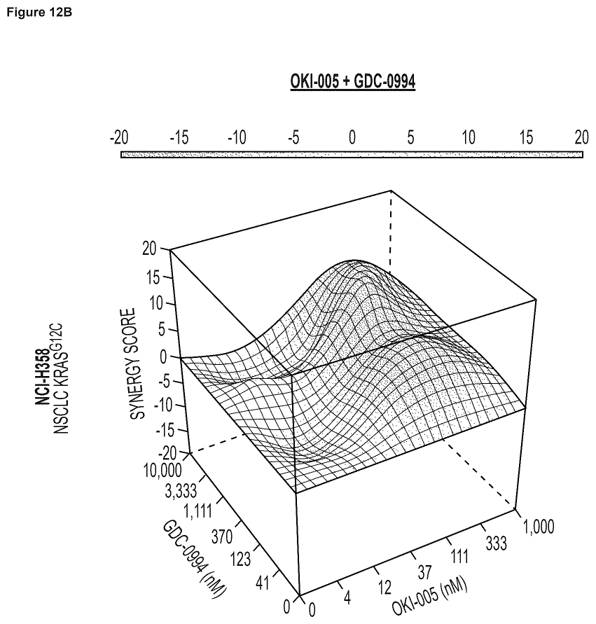

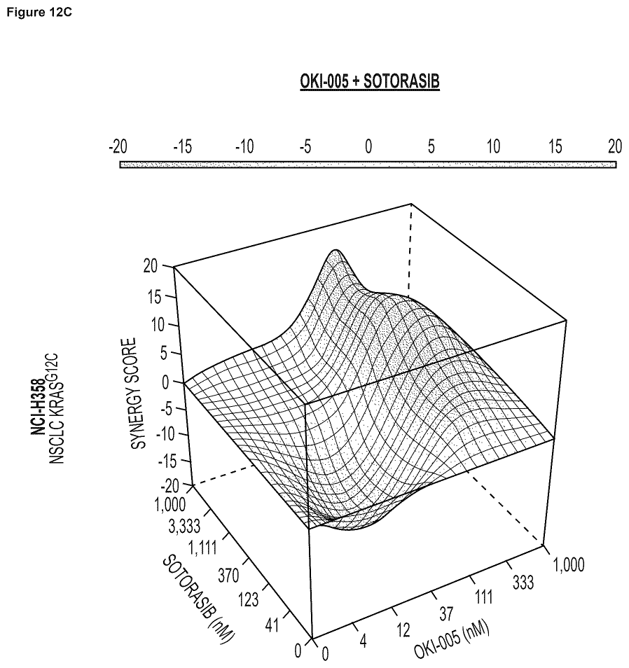

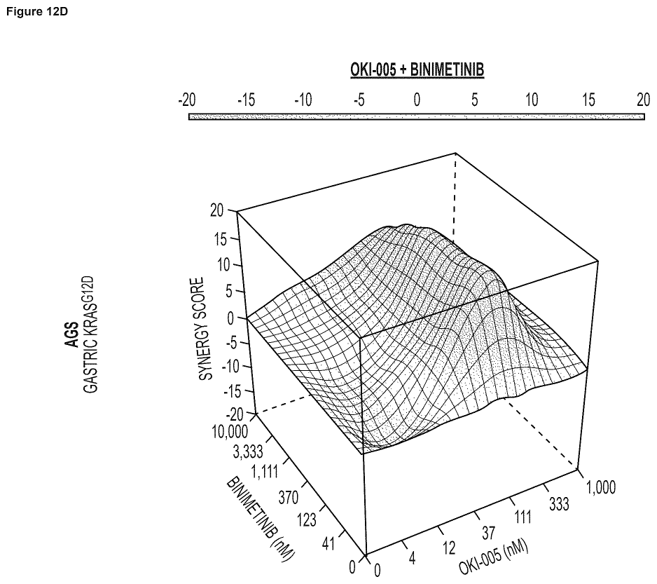

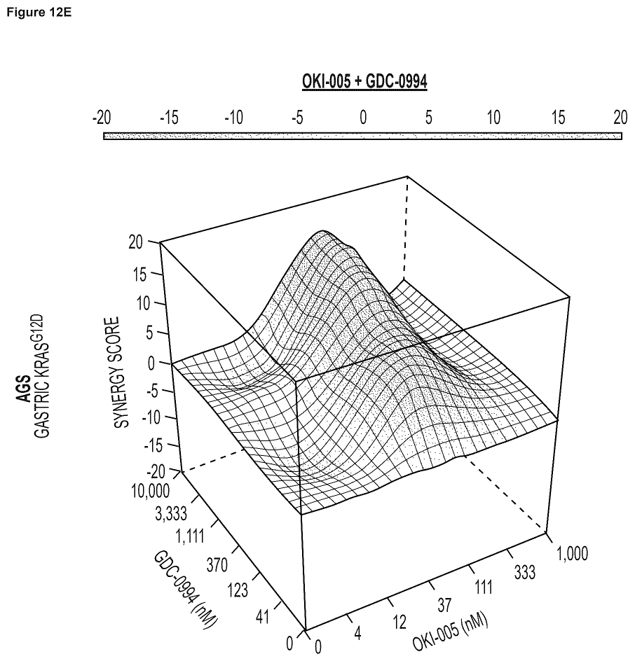

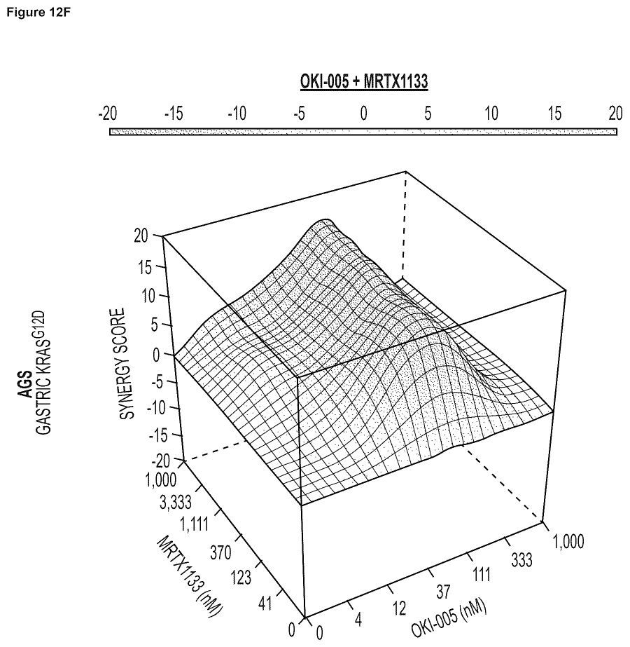

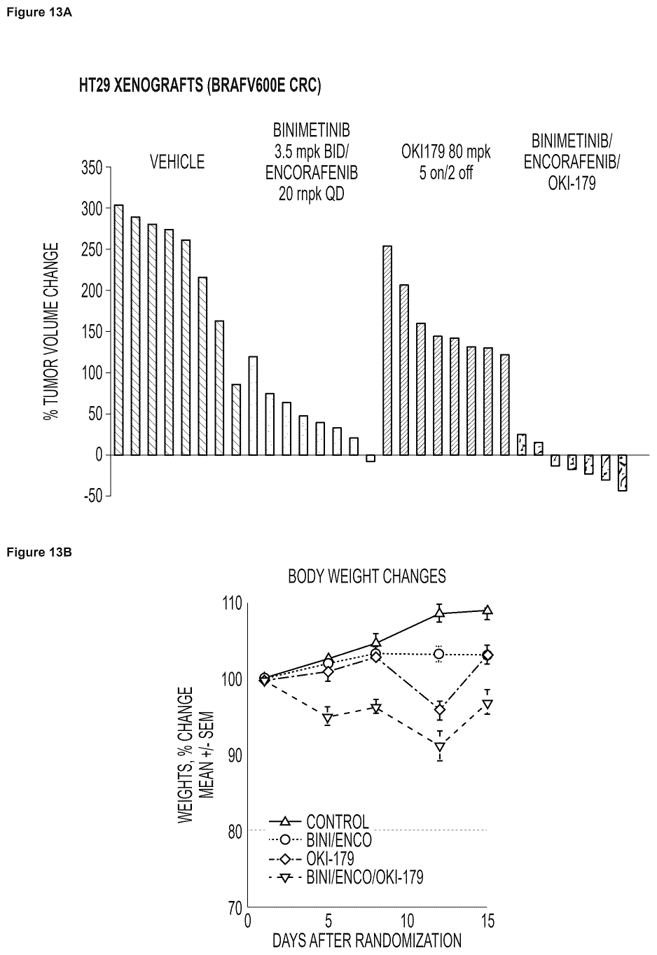

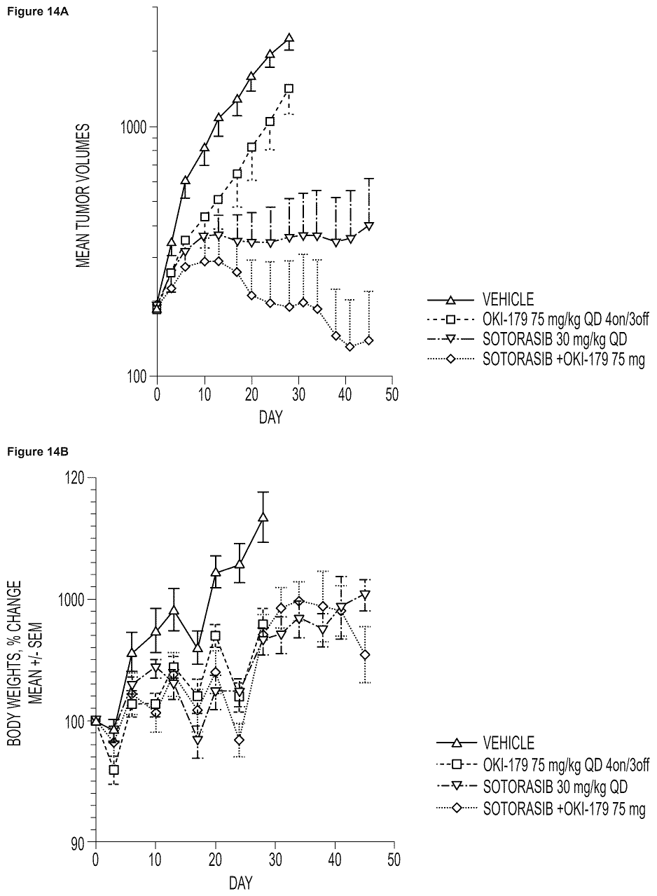

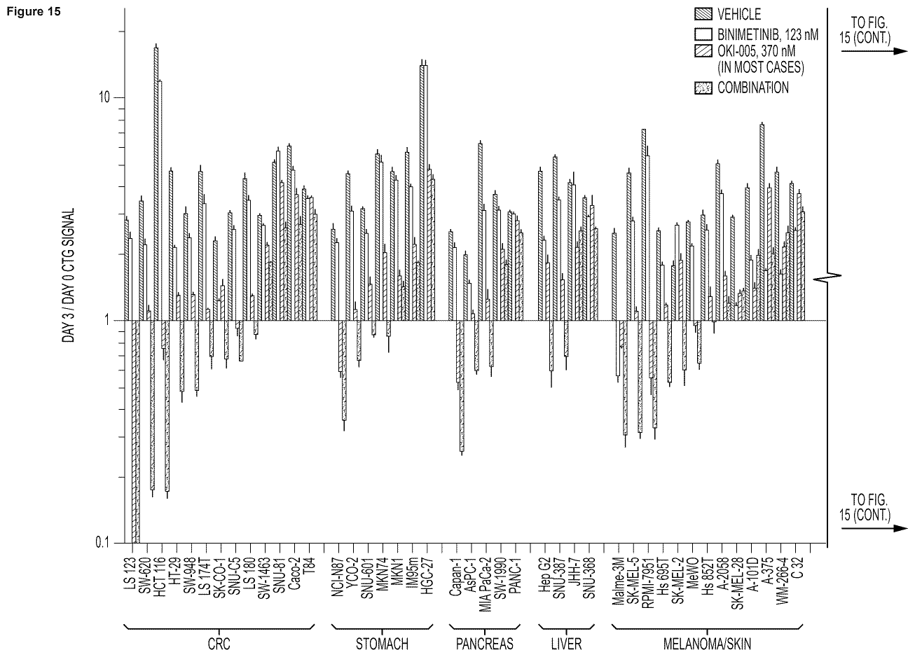

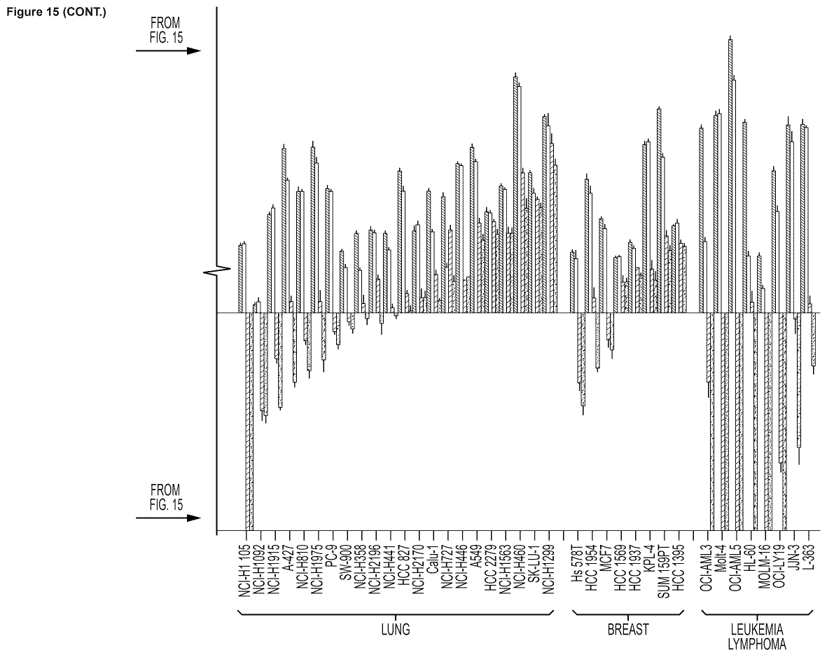

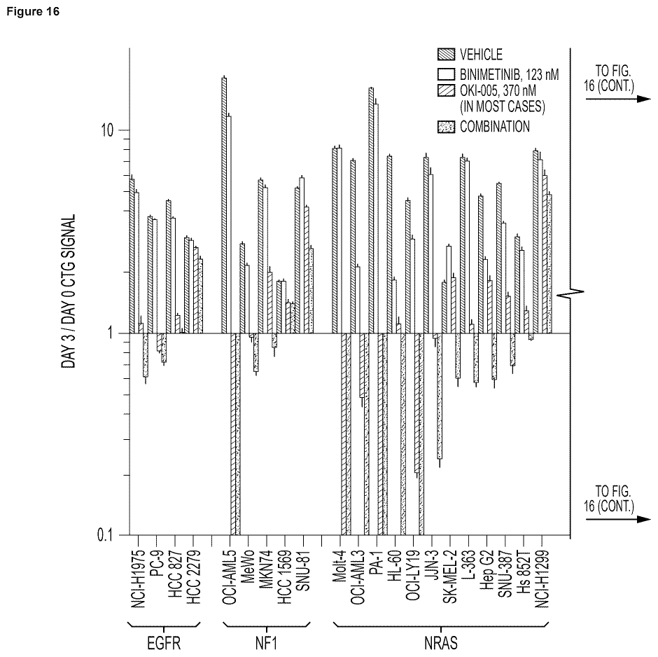

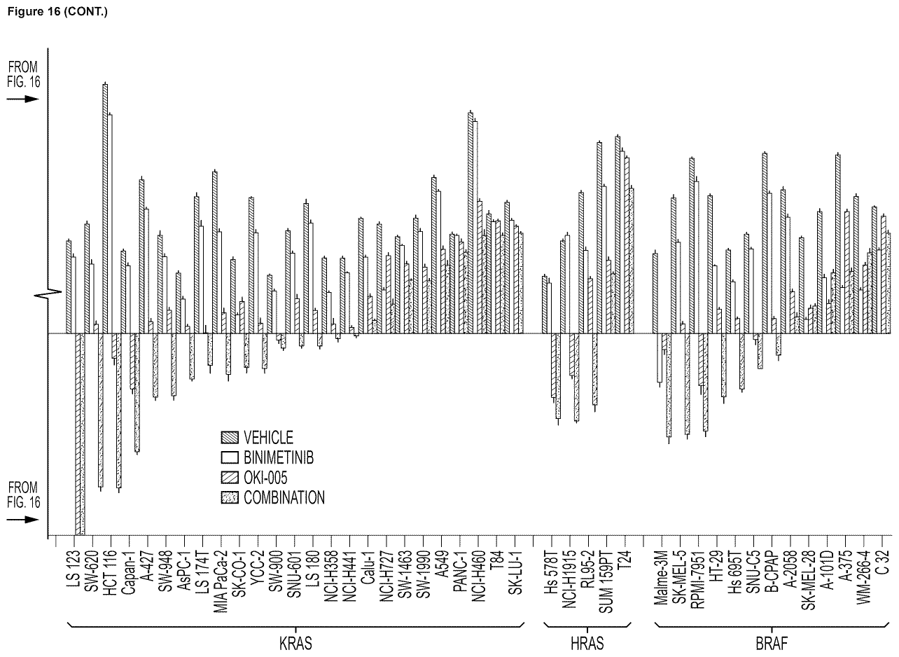

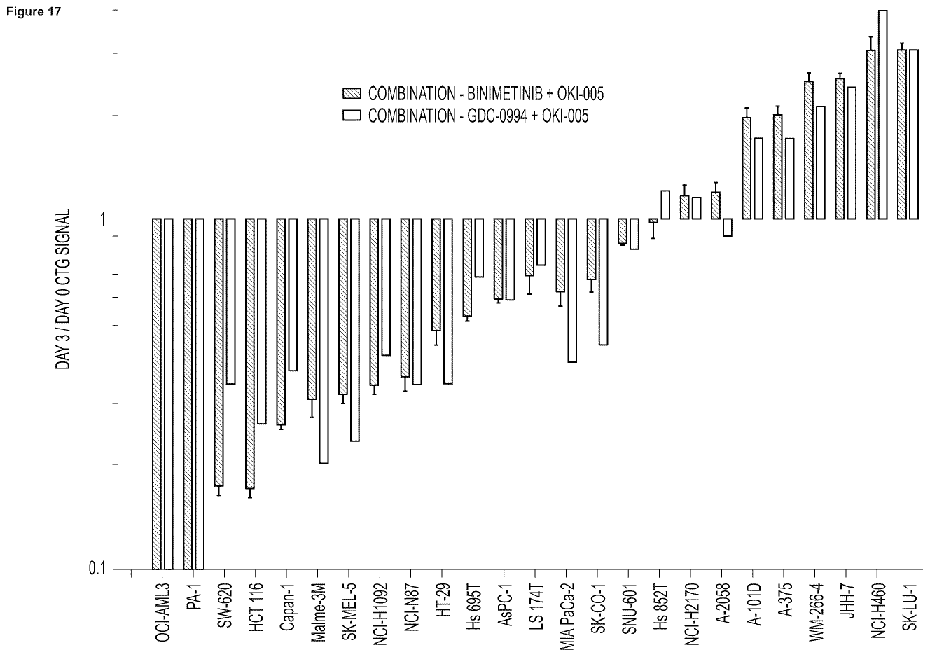

### DETAILED DESCRIPTION OF THE INVENTION

### DEFINITIONS

As defined herein, “OKI-179” refers to the compound of the following structure that exists as a benzenesulfonate salt and is a prodrug of OKI-006.

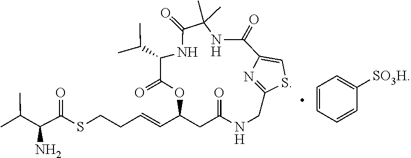 [SMILES: CC(C)[C@H](N)C(=O)SCC/C=C/[C@@H]1CC(=O)NCc2nc(cs2)C(=O)NC(C)(C)C(=O)N[C@@H](C(C)C)C(=O)O1 ; O=S(=O)(O)c1ccccc1]

As defined herein, “OKI-005” refers to the compound of the following structure and is a prodrug of OKI-006.

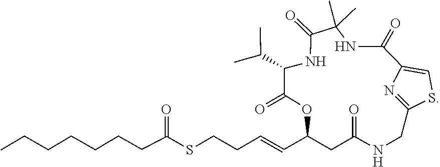 [SMILES: CCCCCCCC(=O)SCC/C=C/[C@@H]1CC(=O)NCc2nc(cs2)C(=O)NC(C)(C)C(=O)N[C@@H](C(C)C)C(=O)O1]

As defined herein, “OKI-006” refers to the compound of the following structure:

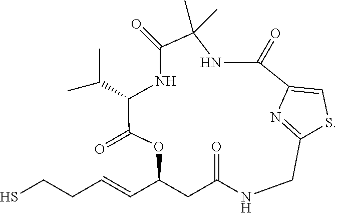 [SMILES: CC(C)[C@@H]1NC(=O)C(C)(C)NC(=O)c2csc(n2)CNC(=O)C[C@@H](/C=C/CCS)OC1=O]

As used herein, various specific inhibitors, such as, but not limited to, MAPK pathway inhibitors, EGFR inhibitors, RAS inhibitors, pan-RAS inhibitors, RAF inhibitors, pan-RAF inhibitors, BRAF inhibitors, MEK inhibitors and ERK inhibitors have their conventional and well known meanings.

As used herein, various specific mutations, such as, but not limited to, mutations in RAS (e.g., KRAS, HRAS or NRAS), mutations in EGFR, mutations in NF1, mutations in BRAF, mutations in MAP2K1, mutations in GNAQ and mutations in GNA11, have their conventional and well known meanings.

As used herein, various specific cell lines, such as, but not limited to, SKMEL2, AGS, MGMT, NCI-H358, uveal melanoma cell lines 92-1 and MP46 have their conventional and well known meanings.

As defined herein, “TGI” refers to tumor growth inhibition.

As defined herein, “R” refers to regression and reflects an observed decrease in tumor size.

As defined herein, “LOF” refers to loss of function.

As defined herein, “BID” refers to a dosage of twice a day.

As defined herein, “QD” refers to a dosage of once a day.

As defined herein, “SEM” refers to standard error of the mean and measures how far the sample mean (average) of the data is likely to be from the true population mean.

As defined herein, the term “subject” includes, but is not limited to, humans (e.g., a male or female of any age group, e.g., a pediatric subject (e.g., infant, child, adolescent) or adult subject (e.g., young adult, middle-aged adult or senior adult)) and/or other primates (e.g., monkeys); non-human mammals, such as cows, pigs, horses, sheep, mice, goats, cats, and/or dogs; and/or birds, such as chickens, ducks and/or geese.

As defined herein, “cancer” refers to a physiological condition in mammals that is typically characterized by unregulated cell growth. Examples of cancer include, but are not limited to, carcinoma, lymphoma, leukemia, blastoma, and sarcoma. More particular examples of such cancers include squamous cell carcinoma, small cell lung cancer, non-small cell lung cancer (NSCLC) (including, but not limited to, metastatic non-small cell lung cancer, BRAF-mutated NSCLC (e.g., BRAF V600E mutated NSCLC), glioma, Hodgkin's lymphoma, non-Hodgkin's lymphoma, acute myeloid leukemia (AML), multiple myeloma, gastrointestinal cancer, renal cell carcinoma, renal cancer (e.g., advanced renal cell carcinoma), ovarian cancer, liver cancer, lymphoblastic leukemia, lymphocytic leukemia, colorectal cancer (including, but not limited to, metastatic colorectal cancer (e.g., microsatellite stable metastatic colorectal cancer), BRAF V600 mutant colorectal cancer (e.g., BRAF V600E or BRAF V600K mutant colorectal cancer), endometrial cancer, kidney cancer, prostate cancer, thyroid cancer, melanoma (including, but not limited to, unresectable or metastatic melanoma, uveal melanoma, BRAF V600 mutant melanoma, such as BRAF V600E mutant melanoma), chondrosarcoma, neuroblastoma, pancreatic cancer, glioblastoma multiforme, cervical cancer, brain cancer, stomach cancer, urothelial carcinoma (including local advanced or metastatic urothelial carcinoma), bladder cancer, hepatoma, breast cancer and head and neck cancer. In one embodiment, the cancer is colorectal cancer. In one embodiment, the cancer is metastatic colorectal cancer. In one embodiment, the cancer is melanoma. In another embodiment, the cancer is pancreatic cancer. In another embodiment, the cancer is NSCLC. In another embodiment, the cancer is BRAF-mutated melanoma. In one embodiment, the cancer is a BRAF-associated cancer. The term “BRAF-associated cancer” as used herein refers to cancers associated with or having either a Class I, Class II or Class III mutation in BRAF (BRAF Class I mutations are mutations at the V600 locus). Class II mutations are non-V600 mutations that activate BRAF by signaling through a RAS-independent dimer. Class III mutations are “kinase-dead” with low kinase activity as compared to wild type BRAF. Non-limiting examples of BRAF-associated cancers are described herein. In one embodiment, the cancer is an KRAS-associated cancer. The term “KRAS-associated cancer” as used herein refers to cancers associated with or having an activating mutation in KRAS, particularly mutations at residues G12, G13 or Q61. Non-limiting examples of KRAS-associated cancers are described herein.

As defined herein, the phrase “combination therapy” refers to refers to a dosing regimen of two or more different therapeutically active agents during a period of time, wherein the therapeutically active agents are administered together or separately in a manner. In one embodiment the combination therapy is a non-fixed combination.

The term “non-fixed combination” means that the two or more different therapeutic agents are formulated as separate compositions or dosages such that they may be administered separately to a subject in need thereof either simultaneously or sequentially with variable intervening time limits.

As defined herein, the phrase “effective dosage” or “effective amount” or “therapeutically effective amount” refers to a particular amount of a pharmaceutical composition comprising a therapeutic agent that achieves a clinically beneficial result (i.e., for example, a reduction of symptoms). Toxicity and therapeutic efficacy of such compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD₅₀/ED₅₀. Compounds that exhibit large therapeutic indices are preferred. The data obtained from these cell culture assays and additional animal studies can be used in formulating a range of dosages for human use. The dosages of such compounds lie preferably within a range of circulating concentrations that include the ED₅₀ with little or no toxicity. The dosage varies within this range depending upon the dosage form employed, the sensitivity of the patient, and the route of administration. In a particular embodiment, OKI-179 is dosed in a range of 100 to 450 mg 4 days on, 3 days off, with the specific dosage being dependent on several factors associated with the subject. In another particular embodiment, OKI-179 is dosed in a range of 100 to 200 mg daily, with the specific dosage being dependent on several factors associated with the subject. In most situations, the recommended clinical dose of the RAS pathway inhibitor should be used in administration of the combination.

The term “disease” as used herein, refers to any impairment of the normal state of the living animal or one of its parts that interrupts or modifies the performance of the vital functions. Typically manifested by distinguishing signs and symptoms, a disease is usually a response to i) environmental factors (such as malnutrition, industrial hazards, or climate); ii) specific infective agents (such as worms, bacteria, or viruses); ill) inherent defects of the organism (such as genetic anomalies); and/or iv) combinations of these factors.

The terms “reduce”, “inhibit”, “diminish”, “suppress”, “decrease”, “prevent” and grammatical equivalents thereof (including “lower”, “smaller”, etc.) when used in reference to the expression of any symptom in an untreated subject relative to a treated subject, indicate that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject,

The term “inhibitory compound” as used herein, refers to any compound capable of interacting with (i.e., for example, attaching, binding, etc.) to a binding partner under conditions such that the binding partner becomes unresponsive to its natural ligands. Inhibitory compounds may include, but are not limited to, small organic molecules, antibodies, and proteins/peptides.

The term “drug” or “compound” or “agent” as used herein, refers to any pharmacologically active substance capable of being administered which achieves a desired effect. Drugs or compounds can be synthetic or naturally occurring, non-peptide, proteins or peptides, oligonucleotides or nucleotides, polysaccharides, or sugars.

The term “administered” or “administering” as used herein, refers to any method of providing a composition to a patient such that the composition has its intended effect on the patient. An exemplary method of administering is by a direct mechanism such as, local tissue administration (i.e., for example, extravascular administration, such as subcutaneous, intramuscular, or intraperitoneal), intravenous, oral ingestion, transdermal patch, topical, inhalation, suppository, etc.

The term “patient” as used herein, is a human or animal and includes both hospitalized and non-hospitalized subjects. For example, out-patients and persons in nursing homes are “patients.” A patient may be a human or non-human animal of any age and therefore includes both adults and juveniles (i.e., children). It is not intended that the term “patient” connote a need for medical treatment. Therefore, a patient may voluntarily be subject to experimentation, whether clinical or in support of basic science studies.

The term “synergy” or “synergistic” as used herein refers to the phenomenon where the combination of two therapeutic agents of a combination therapy is greater in terms of measured results than the sum of the effect of each agent when administered alone.

The term “protein” as used herein, refers to any of numerous naturally occurring extremely complex substances (such as an enzyme or antibody) that contain amino acid residues joined by peptide bonds, and which include carbon, hydrogen, nitrogen, oxygen, and typically sulfur. In general, a protein comprises amino acids having an order of magnitude within the hundreds.

The term “peptide” as used herein, refers to any of various amides that are derived from two or more amino acids by combination of the amino group of one acid with the carboxyl group of another and are usually obtained by partial hydrolysis of proteins. In general, a peptide comprises amino acids having an order of magnitude with the tens.

The term “pharmaceutically acceptable” or “pharmacologically acceptable” as used herein, refers to molecular entities and compositions that do not produce adverse, allergic, or other untoward reactions when administered to an animal or a human.

The term, “pharmaceutically acceptable carrier” as used herein, includes any and all solvents, or a dispersion medium including, but not limited to, water, ethanol, a polyol (such as, for example, glycerol, propylene glycol, and liquid polyethylene glycol, and the like), suitable mixtures thereof, vegetable oils, coatings, isotonic and absorption delaying agents, liposome, commercially available cleansers, and the like. Supplementary bioactive ingredients also can be incorporated into such carriers.

The term “pharmaceutically acceptable salt” as used herein, refers to a salt that does not adversely impact the biological activity and properties of the compound and is suitable for use in contact with the tissues of subjects without undue toxicity, irritation and/or allergic response and the like. Pharmaceutically acceptable salts include those derived from suitable inorganic acids, organic acids and bases, and include hydrochloric acid, hydrobromic acid, phosphoric acid, sulfuric acid, nitric acid, acetic acid, oxalic acid, maleic acid, tartaric acid, citric acid, succinic acid, malonic acid, ascorbic acid, methanesulfonic acid, ethanesulfonic acid, benzenesulfonic acid, p-toluenesulfonic acid, benzoic acid, naphthalene sulfonic acid, lactic acid, succinic acid, oxalic acid, stearic acid, and the like. In some instances, pharmaceutically acceptable salts are obtained by reacting a compound having acidic group described herein with a base to form a salt such as an ammonium salt, an alkali metal salt (e.g., a sodium or a potassium salt), an alkaline earth metal salt (e.g., a calcium or a magnesium salt), a salt formed from an organic base, and an amino acid salt. Pharmaceutically acceptable salts derived from appropriate bases include alkali metals, alkaline earth metals, and ammonium and quaternary ammonium compounds. Specific metals include, but are not limited to, sodium, lithium, potassium, calcium, magnesium, iron, zinc, copper, manganese, aluminum, and the like. Organic bases from which salts may be prepared include, for example, primary, secondary, and tertiary amines.

The term “prodrug” as used herein, refers to a compound that is transformed in vivo to yield a disclosed compound or a pharmaceutically acceptable form of the compound. A prodrug may be inactive when administered to a subject, but is converted in vivo to an active compound. In various instances, a prodrug has improved physicochemical properties (such as bioavailability) and/or delivery properties over the parent compound. Prodrugs are typically designed to enhance pharmaceutically and/or pharmacokinetically based properties associated with the parent compound. The prodrug compound often offers advantages of solubility, tissue compatibility or delayed release in subject. Prodrugs include compounds wherein a hydroxy, amino, or mercapto group is bonded to any group that, when the prodrug is administered to a subject, cleaves to form a free hydroxy, free amino, or free mercapto group, respectively. Prodrugs are well known to be prepared from carboxylic acids in the form of, for example, carboxylate esters or thioesters.

The term “biologically active” as used herein, refers to any molecule having structural, regulatory or biochemical functions. For example, biological activity may be determined, for example, by restoration of wild-type growth in cells lacking protein activity. Cells lacking protein activity may be produced by many methods (i.e., for example, point mutation and frame-shift mutation). Complementation is achieved by transfecting cells which lack protein activity with an expression vector which expresses the protein, a derivative thereof, or a portion thereof.

The term “label” or “detectable label” as used herein, refers to any composition detectable by spectroscopic, photochemical, biochemical, immunochemical, electrical, optical or chemical means. Such labels include biotin for staining with labeled streptavidin conjugate, magnetic beads (e.g., Dynabeads®), fluorescent dyes (e.g., fluorescein, Texas Red®, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold or colored glass or plastic (e.g., polystyrene, polypropylene, latex, etc.) beads. Patents teaching the use of such labels include, but are not limited to, U.S. Pat. Nos. 3,817,837; 3,850,752; 3,939,350; 3,996,345; 4,277,437; 4,275,149; and 4,366,241 (all herein incorporated by reference in their entireties). The labels contemplated in the present invention may be detected by conventional methods. For example, radiolabels may be detected using photographic film or scintillation counters, fluorescent markers may be detected using a photodetector to detect emitted light. Enzymatic labels are typically detected by providing the enzyme with a substrate and detecting, the reaction product produced by the action of the enzyme on the substrate, and calorimetric labels are detected by simply visualizing the colored label.

Pharmaceutical compositions of the invention can take any suitable form for the desired route of administration. Where the composition is to be administered orally, any suitable orally deliverable dosage form can be used, including without limitation water, glycols, oils, alcohols, and the like in the case of oral liquid preparations such as suspensions, syrups, elixirs, emulsions, and solutions; or solid carriers such as starches, sugars, kaolin, diluents, lubricants, binders, disintegrating agents, and the like in the case of powders, pills, capsules, and tablets. Because of their ease in administration, tablets and capsules represent the most advantageous oral dosage unit forms. Injectable compositions or intravenous infusions are also provided in the form of solutions, suspensions, and emulsions. For parenteral compositions, the carrier usually comprises sterile water and possibly other ingredients to aid solubility. Injectable solutions may be prepared in which the carrier comprises a saline solution, a glucose solution, or a mixture of a saline and a glucose solution. Suitable oils include, for example, peanut oil, sesame oil, cottonseed oil, corn oil, soybean oil, synthetic glycerol esters of long chain fatty acids, and mixtures of these and other oils. In compositions suitable for percutaneous administration, the carrier optionally comprises a penetration enhancing agent and/or a suitable wetting agent, optionally combined with suitable additives as needed, where the additives may facilitate administration of the composition to the skin and/or may facilitate preparation of the compositions to be delivered. These compositions may be administered in various ways, e.g., as a transdermal patch or as an ointment. Acid or base addition salts of the compounds of the invention are typically more suitable in the preparation of aqueous compositions due to their increased water solubility over the corresponding neutral form of the compounds.

Pharmaceutical compositions of the invention may comprise one or more of a filler, diluent, adjuvant, vehicle, or other excipient to facilitate storage and/or administration of the active ingredients contained therein.

In an exemplary embodiment, a pharmaceutical composition according to the present invention may contain one or more additional therapeutic agents, for example, to increase efficacy or to decrease undesired side effects. Examples of such agents include, without limitation, agents to treat or inhibit cancer, Huntington's disease, cystic fibrosis, liver fibrosis, renal fibrosis, pulmonary fibrosis, skin fibrosis, rheumatoid arthritis, diabetes, or heart failure.

In a specific embodiment, the additional therapeutic agent to be included is an anti-cancer agent. Examples of an anti-cancer agent include, but are not limited to, MAPK pathway inhibitors, alkylating agents such as cyclophosphamide, dacarbazine, and cisplatin; anti-metabolites such as methotrexate, mercaptopurine, thioguanine, fluorouracil, and cytarabine; plant alkaloids such as vinblastine and paclitaxel; antitumor antibiotics such as doxorubicin, bleomycin and mitomycin; hormones/antihormones such as prednisone, tamoxifen, and flutamide; other types of anticancer agents such as asparaginase, rituximab, trastuzumab, imatinib, retinoic acid, and derivatives, colony stimulating factors, amifostine, camptothecin, topotecan, thalidomide analogs such as lenalidomide, CDK inhibitors, and proteasome inhibitors such as Velcade.

In another embodiment, the present invention provides a method of inhibiting or treating diseases arising from abnormal cell proliferation and/or differentiation in a subject in need thereof, comprising administering to the subject a therapeutically effective amount of one or more compounds according to the present invention. In one embodiment, the method of inhibiting or treating disease comprises administering to a subject in need thereof, a composition comprising an effective amount of one or more compounds of the invention and a pharmaceutically acceptable carrier. The composition to be administered may further contain a therapeutic agent such as an anti-cancer agent.

The present invention includes compounds labeled with various radioactive or nonradioactive isotopes. Examples of atomic isotopes may include, but are not limited to, deuterium (²H), tritium (³H), iodine-125 (¹²⁵I), carbon-14 (¹⁴C), nitrogen-15 (¹⁵N), sulfur-35 (³⁵S) and chlorine-36 (³⁶Cl). In an exemplary embodiment, one or more hydrogen atoms in a compound of the invention can be replaced by deuterium. In various embodiments, a compound of the invention includes at least one deuterium atom, or two or more deuterium atoms, or three or more deuterium atoms, etc. As described herein, compounds of the invention may also be radiolabeled with a radioactive isotope such as tritium (3H), iodine-125 (¹²⁵I), and carbon-14 (¹⁴C). A radiolabeled compound is useful as a therapeutic or prophylactic agent, provides a reagent for research such as for an assay, and/or provides a diagnostic agent for techniques such as in vivo imaging. Synthetic methods for incorporating isotopes into organic compounds are well known in the art.

As defined herein, the term “in vivo” refers to an event that takes place in a subject's body.

As defined herein, the term “in vitro” refers to an event that takes places outside of a subject's body.

As defined herein, a “pharmaceutically acceptable form” of a compound includes, but is not limited to, pharmaceutically acceptable salts, hydrates, solvates, isomers, prodrugs, and isotopically labeled derivatives of a compound.

As defined herein, the phrase “treat” or “treating” a cancer means to administer a compound of the present invention to a subject having cancer or having been diagnosed with cancer, to achieve at least one positive therapeutic effect, such as, for example, reduced number of cancer cells, reduced tumor size, reduced rate of cancer cell infiltration into peripheral organs, or reduced rate of tumor metastases or tumor growth, reversing, alleviating, or inhibiting the progress of, the disorder or condition to which such term applies, or one or more symptoms of such disorder or condition. The term “treatment”, as used herein, unless otherwise indicated, refers to the act of treating as “treating” is defined immediately above. The term “treating” also includes adjuvant and neo-adjuvant treatment of a subject.

For the purposes of this invention, beneficial or desired clinical results include, but are not limited to, one or more of the following: reducing the proliferation of (or destroying) neoplastic or cancerous cell; inhibiting metastasis or neoplastic cells; shrinking or decreasing the size of a tumor; an increase in the duration of response, progression free survival or overall survival for a subject (e.g., as compared to the one or more metric(s) in a subject having a similar cancer receiving no treatment or a different treatment, or as compared to the one or more metric(s) in the same subject prior to treatment); decreasing symptoms resulting from the cancer; increasing the quality of life of those suffering from the cancer; decreasing the dose of other medications required to treat the cancer; delaying the progression of the cancer; curing the cancer; overcoming one or more resistance mechanisms of the cancer; and/or prolonging survival of patients the cancer. Positive therapeutic effects in cancer can be measured in several ways (see, e.g., W. A. Weber, J. Nucl. Med. 50 Suppl. 1:1 S-10S (2009)).

The mitogen-activated protein kinase (MAPK) pathway (also known as the Ras-Raf-MEK-ERK pathway) is a chain of proteins in the cell that communicates a signal from a receptor on the surface of the cell to the DNA in the nucleus of the cell. The MAPK pathway controls many fundamental cellular processes related to proliferation, differentiation and survival of the cell. In normal cells, RAS activates RAF kinases which in turn phosphorylate and activate MEK1 and MEK2, which upon activation, phosphorylate ERK1 and ERK2. ERK regulates the activity and expression of multiple nuclear transcription factors and cytosolic proteins required for cell proliferation, differentiation and survival. There are a number of mutations in the MAPK pathway proteins that effectively de-regulate the pathway in several human cancers and promote cell survival and proliferation of cancer or tumor cells.

Examples of MAPK pathway inhibitors include, but are not limited to, EGFR inhibitors, MEK inhibitors, ERK inhibitors, BRAF inhibitors, pan-RAF inhibitors, KRAS inhibitors, pan-RAS inhibitors and RAF inhibitors.

Exemplary RAF inhibitors include, but are not limited to, RAF265, sorafenib, dabrafenib (GSK2118436), 5B590885, PLX 4720, PLX4032, GDC-0879 and ZM 336372.

Exemplary MEK inhibitors include, but are not limited to, trametinib (GSK1120212), selumetinib, binimetinib, cobinmetinib, PD-325901, CI-1040/PD184352, TAK-733, AZD6244, PD318088, PD98059, PD334581 and RDEA119.

Exemplary KRAS inhibitors include, but are not limited to, sotorasib, adagrasib, RMC-6236, RMC-6291, RMC-9805 and RMC-8839.

Exemplary ERK inhibitors include, but are not limited to, ulixertinib, VTX11e, AEZS-131, PD98059, FR180204 and FR148083.

The mammalian HDACs can be divided into four classes depending on their sequence homology, sub-cellular distribution and catalytic activity. Class I, Il and IV HDACs have a Zn²⁺ cofactor in the catalytic site. Class III HDACs contain sirtuins 1-7 and are NAD⁺-dependent. Class I (HDACs 1, 2, 3 and 8) share the same homology as Rpd3 and are preliminarily distributed in the nucleus of normal cells. Class II HDACs can be subdivided into class IIa HDACs and class Ilb HDACs. Class IIa enzymes (HDAC 4, 5, 7 and 9) shuttle between the nucleus and cytoplasm and are located in specific tissues such as the brain, heart, and muscle. Class IIb (HDACs 6 and 10) are chiefly located in the cytoplasm. Class IV only contains HDAC 11 and is located in the brain, heart, kidneys, skeletal muscle and testis, Below is a table showing the enzymatic inhibition of HDAC 1 through HDAC 11 by known HDAC inhibitors Vorinostat and Romidepsin (FK228) compared to OKI-006, where OKI-006 exhibits high potency for Class I HDAC inhibitors.

Compound IC50 (nM)

| Class | HDAC | OKI-006* | Red-FK228** | Vorinostat |
|---|---|---|---|---|
| I | 1 | 1.2 | 0.8 | 75.5 |
| I | 2 | 2.4 | 1.0 | 362.0 |
| I | 3 | 2.0 | 1.3 | 57.4 |
| I | 8 | 49 | >1000 | 1069 |
| IIa | 4 | >1000 | 647.0 | >1000 |
| IIa | 5 | >1000 | >1000 | 163 |
| IIa | 7 | >1000 | >1000 | >1000 |
| IIa | 9 | >1000 | >1000 | 78.1 |
| IIb | 6 | 47.0 | 226.0 | 27.1 |
| IIb | 10 | 2.8 | 0.9 | 88.4 |
| IV | 11 | 2.3 | 0.3 | 109.0 |

*OKI-006 is the active form of OKI-179
**Red-FK228 is the active (reduced) form of romidepsin

Suitable dosages of OKI-179 alone and in combination with other inhibitors can be determined by one skilled in the art. It will be appreciated that appropriate dosages can vary from patient to patient. Determining the optimal dosage will generally involve the balancing of the level of therapeutic benefit against any risk or deleterious side effects of the treatments described herein. The selected dosage level will depend on a variety of factors including, but not limited to, the activity of the particular compound, the route of administration, the time of administration, the rate of excretion of the compound, the duration of the treatment, other drugs, compounds, or materials used in combination, and the age, sex, weight, condition, general health, and prior medical history of the patient. The amount(s) of the compound(s) administered and the route of administration will ultimately be at the discretion of a physician. Administration of OKI-179 alone or in combination with another inhibitor may occur in one dose, continuously or intermittently (e.g., in divided doses at appropriate intervals) throughout the course of treatment. The following represents 3 exemplary schedules for administering OKI-179 that have achieved a maximally-tolerated dose (MTD) in advanced cancer patients when administered orally. The MTD for OKI-179 on Schedule 1 was 450 mg daily for 4 days on and 3 days off each week where the recommended Phase 2 dose (RP2D) was 300 mg. On Schedule 2, the MTD was 200 mg daily on a continuous dosing regimen and on Schedule 3, the MTD was 300 mg for 5 days on and 2 days off each week.

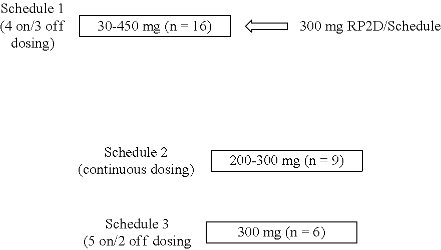

As an example of the frequency of mutations associated with various cancers, mutations in NRAS account for 20% of all melanomas and affect approximately 65,000 patients worldwide. Such melanomas have a tendency to be more aggressive than their wild-type counterparts and frequently have poor outcomes. Current therapies for NRAS-mutant melanoma remain limited, with little efficacy. No targeted therapies are currently available post PD1i. Preclinical activity of OKI-179 in combination with binimetinib shows regressions in multiple RAS-pathway mutated models. The below data exemplifies the effectiveness of the combination of OKI-179 with binimetinib against specific mutated tumor types compared to their wild-type counterparts, where “N” is the number of conventional models used for the indicated tumor type, and “GI” is gastrointestinal,

Ras Pathway Status
(% Combo Cell KiII)

| Tumor Type | N | Mutated | Wild-Type |
|---|---|---|---|
| Colon | 12 | 81% (9/11) | 0% (0/1) |
| Lung | 20 | 54% (7/13) | 43% (3/7) |
| Melanoma | 11 | 55% (6/11) | — |
| Other GI | 13 | 89% (8/9) | 0% (0/4) |
| ALL | 56 | 68% (30/44) | 25% (3/12) |

The methods of this invention may be carried out for the treatment of patients with cancers that harbor a mutation that results in the activation of the RAS pathway. For example, a physician would test for mutations in genes in the RAS pathway (such as EGFR RAS, RAF, MAP2K1, NF1 or GNAQ/11) using available mutation-testing methodologies as a means of assessing suitability for the combination of OKI-179 with a RAS-pathway inhibitor. In an exemplary embodiment, OKI-179 and a RAS-pathway inhibitor are co-administered to patients with tumors that have an activating mutation in EGFR, RAS, RAF, MAP2K1, NF1 or GNAQ/11, but this combination would not be used in patients without a RAS-pathway activating mutation. The doses and schedules of OKI-179 and the RAS pathway inhibitor would be recognized as tolerated and effective by physicians. The therapeutic effects of the respective combinations may be determined using established methods and approaches to show their impact on tumor shrinkage and/or a survival outcome in patients.

1. A method of treating cancer in a subject comprising administering to the subject in need thereof therapeutically effective amounts of
OKI-179; and
one or more mitogen-activated protein kinase (MAPK) pathway inhibitors,
wherein the cancer harbors one or more mutations that result in activation of the MAPK pathway.

2. The method according to claim 1, wherein administration of the OKI-179 and the one or more MAPK pathway inhibitors in combination results in synergistic inhibition of cancer growth.

3. The method according to claim 1, wherein the one or more MAPK pathway inhibitors is selected from an EGFR inhibitor, a RAS inhibitor, a BRAF inhibitor, a pan-RAF inhibitor, a MEK inhibitor and an ERK inhibitor.

4. The method according to claim 1, wherein the mutation occurs in one or more of EGFR, NF1, RAS, BRAF, MAP2K1, GNAQ and GNA11.

5. The method according to claim 1, wherein the one or more MAPK pathway inhibitors comprises an EGFR inhibitor.

6. The method according to claim 1, wherein the one or more MAPK pathway inhibitors comprises a RAS inhibitor.

7. The method according to claim 1, wherein the one or more MAPK pathway inhibitors comprises a BRAF inhibitor.

8. The method according to claim 1, wherein the one or more MAPK pathway inhibitors comprises a pan-RAF inhibitor.

9. The method according to claim 1, wherein the one or more MAPK pathway inhibitors comprises a MEK inhibitor.

10. The method according to claim 1, wherein the one or more MAPK pathway inhibitors comprises an ERK inhibitor.

11. The method according to claim 1, wherein the mutation occurs in two or more of EGFR, NF1, RAS, BRAF, MAP2K1, GNAQ and GNA11.

12. The method according to claim 1, wherein the mutation occurs in three or more of EGFR, NF1, RAS, BRAF, MAP2K1, GNAQ and GNA11.

13. The method according to claim 1, wherein the at least one MAPK pathway inhibitor and the OKI-179 are administered concurrently.

14. The method according to claim 1, wherein the at least one MAPK pathway inhibitor and the OKI-179 are administered sequentially.

15. The method according to claim 1, wherein the cancer is colorectal cancer.

16. The method according to claim 1, wherein the cancer is breast cancer.

17. The method according to claim 1, wherein the cancer is non-small lung cancer.

18. The method according to claim 1, wherein the cancer is melanoma.

19. A pharmaceutical combination for the treatment of cancer, the combination comprising:
OKI-179; and
one or more mitogen-activated protein kinase (MAPK) pathway inhibitors,
wherein the cancer harbors one or more mutations that result in activation of the MAPK pathway.

20. The combination according to claim 19, wherein the one or more MAPK pathway inhibitors is selected from an EGFR inhibitor, a RAS inhibitor, a BRAF inhibitor, a pan-RAF inhibitor, a MEK inhibitor and an ERK inhibitor.

21. The combination according to claim 19, wherein administration of the combination of the OKI-179 and the one or more MAPK pathway inhibitors results in synergistic inhibition of cancer growth.

22. The combination according to claim 19, wherein the mutation occurs in one or more of EGFR, NF1, RAS and BRAF, MAP2K1, GNAQ or GNA11.
